(19) United States
(12) Patent Application Publication
(10) Pub. No.: US 2022/0315517 A1
(43) Pub. Date: Oct. 6, 2022

(54) METHOD FOR OXIDATIVE CLEAVAGE OF OLEFINS USING A HALOOXODIPEROXOMETALLATE AS A CATALYST

(71) Applicants: DEMETA, RENNES (FR); UNIVERSITE DE RENNES 1, RENNES (FR)

(72) Inventors: CORENTIN BORDIER, SAINT-CALAIS (FR); VINCENT ESCANDE, RENNES (FR); CHRISTOPHE DARCEL, RENNES (FR); FRÉDÉRIC CAIJO, THORIGNE FOUILLARD (FR)

(21) Appl. No.: 17/642,248

(22) PCT Filed: Sep. 14, 2020
(86) PCT No.: PCT/FR2020/051585

§ 371 Date: Mar. 11, 2022

(30) Foreign Application Priority Data
Sep. 16, 2019 (FR) FR1910191

(51) Int. Cl. C07C 51/25; B01J 31/02

(52) U.S. Cl. CPC C07C 51/25; B01J 31/0239; B01J 2231/70; B01J 2531/64; B01J 2531/66

(57) ABSTRACT

The present invention relates to a method for oxidative cleavage of a substrate consisting of at least one functionalised or non-functionalised linear olefin, in particular a mono- or polyunsaturated aliphatic carboxylic acid, or one of the esters thereof, or at least one non-functionalised cyclic olefin, using hydrogen peroxide, in the presence of a metal catalyst which consists of at least one onium halooxodiperoxometallate. It also relates to a novel catalyst consisting of a specific onium halooxodiperoxometallate which can be used, in particular, in said method.

### SUBJECT-MATTER OF THE INVENTION

The present invention relates to a process for the oxidative cleavage of a substrate consisting of at least one functionalized or non-functionalized linear olefin, in particular of a mono- or polyunsaturated aliphatic carboxylic acid, or one of its esters, or of at least one non-functionalized cyclic olefin, using hydrogen peroxide, in the presence of a metal catalyst, which is formed of at least one onium halooxodiperoxometallate. Another subject-matter is a novel catalyst consisting of a specific onium halooxodiperoxometallate which can in particular be employed in this process.

### BACKGROUND OF THE INVENTION

Known for many years and always forming the subject of new developments, the oxidative cleavage of olefins is a chemical reaction which makes possible the conversion of a carbon-carbon double bond into two separate oxidized functional groups, such as aldehydes, ketones or carboxylic acids. This reaction is very particularly advantageous in the upgrading of vegetable oils. This is because oxidative cleavage converts unsaturated fatty acids in one stage into high-added-value oxidation products, used both in the polymer industry and in the food-processing industry or even the perfumery industry. For example, oleic acid is converted by oxidative cleavage into pelargonic acid and azelaic acid. Azelaic acid, or nonanedioic acid, is a dicarboxylic acid used as precursor in the manufacture of polymers, such as polyesters or polyamides, or in the manufacture of lubricants. This compound is also advantageous as cosmetic and dermatological active agent, due to its antimicrobial properties. For its part, pelargonic acid, or nonanoic acid, is a carboxylic acid which can be used as herbicide, alone or in combination with azelaic acid, or also as emollient agent.

Ozonolysis, which uses ozone O₃ as oxidizing agent, is the most widely employed process for the oxidative cleavage of olefins. Although this process is clean and efficient, the use of ozone requires the implementation of strict safety measures, as well as the installation of expensive equipment. The chemical industry has thus turned to the development of catalytic systems based on transition metals and on oxidizing agents which are less toxic and dangerous. Several processes using catalysts based on precious metals, such as rhenium, ruthenium and gold, have been developed (WO 2014/020281). However, the high catalytic loads and the absence of recycling of these expensive catalysts make it difficult to envisage the development of such processes at the industrial level. In combination with hydrogen peroxide, a relatively inexpensive oxidizing agent, molybdenum and tungsten have also demonstrated their potential in the oxidative cleavage of olefins.

The patent U.S. Pat. No. 5,336,793 thus describes a process for the preparation of carboxylic acids or esters by oxidative cleavage of unsaturated esters or acids in a two-phase medium, in which the organic phase contains the reactants and the aqueous phase includes hydrogen peroxide and a catalyst consisting of tungstic or molybdic acid. The process is characterized by the addition of an onium salt, such as tetraalkylammonium or tetraalkylphosphonium chloride, which acts as phase transfer agent, making it possible to bring the catalyst into contact with the reactants and thus resulting in an improvement in the yield without requiring the use of an organic solvent. The reaction conditions employed in this patent are, however, incompatible with an industrial application. This is because the reaction products are extracted using ethyl ether in the presence of aqueous hydrogen peroxide solution and then the solvent is evaporated, potentially resulting in the formation of diethyl peroxides in the concentrated state and thus of a highly explosive mixture.

The document WO 2013/093366 describes another process for the synthesis of azelaic acid and pelargonic acid by oxidative cleavage of oleic acid, in which the reaction is carried out in a single stage, comprising the in situ formation of a catalyst consisting of a quaternary ammonium salt of phosphotungstic acid, for the purpose of increasing the yield of the reaction. However, the catalytic load by weight used is 19% by weight, which constitutes a value prohibitive for an industrial process, in view of the high price of phosphotungstic acid.

There thus remains a real need to develop an efficient process for the synthesis of dicarboxylic acid by oxidative cleavage of olefins, making it possible to obtain this dicarboxylic acid under industrial conditions which are satisfactory from the viewpoint of economics and of the safety of the process. More generally, the need remains to have available a catalyst which is effective in the oxidative cleavage of a variety of olefins.

In this context, the inventors have developed a process for the oxidative cleavage of olefins using, as catalyst, an onium salt of a halooxodiperoxometallate. A compound of this type has already been described in the publication by Ryo Ishimoto et al., Chem. Lett., 2013, 42, 476-478, where it is used as precursor in the manufacture of a catalyst for the selective oxidation of alkenes. The patent application EP 0 122 804 also mentions a compound obtained by reacting an onium halide with tungstic acid or a salt, in the presence of hydrogen peroxide at a pH of less than 2, in particular at a pH of 1, of which it has now been demonstrated that it corresponds to an onium halooxodiperoxotungstate. In the application EP 0 122 804, this compound is used as catalyst in the oxidative cleavage of diols, as an alternative to an onium phosphotungstate.

However, it is indicated that this alternative provides lower yields of carboxylic acids, unless p-tert-butylphenol is added to the reaction medium. In point of fact, this compound has recently been listed as a potential endocrine disruptor by the European Commission. The patent application JP 2013/144626 also describes the use of mononuclear catalysts, in particular an onium halooxodiperoxometallate, or binuclear catalysts, in combination with hydrogen peroxide in an olefin epoxidation process. However, very low yields of epoxides are obtained with the mononuclear catalysts.

It thus remains necessary to provide a process involving less toxic reactants in order to produce a dicarboxylic acid with good yields.

Surprisingly, it has now been demonstrated that this need can be satisfied by using the alternative catalyst prepared as described in EP 0 122 804 in the oxidative cleavage of olefins. This is all the more surprising since this oxidative cleavage process involves a cascade of reactions, comprising the epoxidation of the substrate, and the reaction of the epoxide obtained to form a hydroperoxyalcohol and/or an α-diol, subsequently resulting in the formation of aldehydes which are finally oxidized to give acids. It was thus not obvious that a single catalyst may be effective throughout these transformations or in any case not interfere negatively with one of them.

### SUMMARY OF THE INVENTION

A subject-matter of the invention is thus a process for the oxidative cleavage of a substrate consisting of at least one functionalized or non-functionalized linear olefin or of at least one non-functionalized cyclic olefin, consisting in converting a carbon-carbon double bond of the substrate into two separate oxidized functional groups chosen from aldehydes, ketones and carboxylic acids, using hydrogen peroxide, in the presence of a metal catalyst, characterized in that the catalyst is formed of at least one onium salt of a halooxodiperoxometallate.

Another subject-matter of the invention is novel catalysts of formula (II):

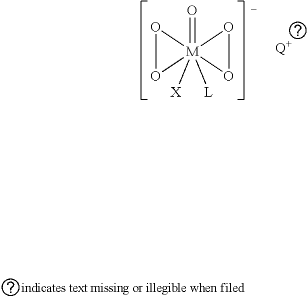

in which:

M is a metal chosen from W and Mo,

X is a halogen atom,

L denotes a neutral ligand having at least one non-bonding lone pair,
Q⁺ denotes an onium cation of formula N⁺(R₁R₂R₃R₄), where:
   R₁ denotes a linear or branched, preferably linear, C₆-C₂₀ alkyl group, R₂ and R₃ each independently denote a linear or branched, preferably linear, C₁-C₄ alkyl group and R₄ denotes a linear or branched, preferably linear, C₁-C₄ alkyl group or an aryl group, or else
   R₁ denotes a linear or branched, preferably linear, C₄-C₁₄ alkyl group and R₂, R₃ and R₄ form, with the nitrogen atom, a pyridinium group.

Another subject-matter of the invention is the use of this catalyst in the oxidative cleavage of mono- or polyunsaturated aliphatic carboxylic acids or their esters.

### DETAILED DESCRIPTION

In one embodiment, the oxidative cleavage process according to the invention uses, as substrate, at least one functionalized or non-functionalized linear olefin. The term “non-functionalized olefin” is understood to mean a hydrocarbon chain including only carbon and hydrogen atoms and which comprises at least one unsaturation. The term “functionalized olefin” is understood to mean a hydrocarbon chain including carbon and hydrogen atoms, comprising at least one unsaturation, and additionally carrying at least one, and generally from one to four, group(s) which is (are) inert under the conditions of the oxidative cleavage reaction, in particular chosen from: a carboxyl (—COOH) group, an alkoxycarbonyl (—OCOR) group, a hydroxyl (—OH) group, a nitro (—NO₂) group, a halogen (in particular —Cl or —F) atom, an alkoxy (—OR) group, an alkylcarbonyl (—COR) group, an amido (—CONH₂) or dialkylamino (—NR₂) group or a nitrile (—CN) group, where R denotes a hydrocarbon group including from one to nine carbon atoms.

The oxidative cleavage process according to the invention consists in converting a carbon-carbon double bond of the substrate into two separate oxidized functional groups, and thus makes it possible to prepare carbonyl compounds of aldehydes, ketones and/or carboxylic acids type, and more particularly mono-, di- and/or tricarboxylic acids.

In a preferred embodiment of the invention, the olefin is functionalized by at least one carboxyl or alkoxycarbonyl group. The functionalized linear olefin is thus chosen from mono- or polyunsaturated aliphatic carboxylic acids and their esters. This carboxylic acid can include from 6 to 60 carbon atoms, preferably from 6 to 32 carbon atoms, more preferentially from 12 to 24 carbon atoms and more preferentially still from 12 to 18 carbon atoms and it can comprise from 1 to 6 unsaturations, preferably from 1 to 3 unsaturations.

Examples of mono- or polyunsaturated aliphatic carboxylic acid comprise lauroleic acid, myristoleic acid, palmitoleic acid, sapienic acid, petroselaidic acid, oleic acid, elaidic acid, petroselinic acid, vaccenic acid, gadoleic acid, cetoleic acid, erucic acid, selacholeic or nervonic acid, α-linoleic acid, γ-linolenic acid, rumenic acid, linolenic acid, stearidonic acid, eleostearic acid, catalpic acid, arachidonic acid and their mixtures. The acid can optionally be mono- or polyhydroxylated and chosen in particular from ricinoleic acid. The abovementioned acid can be obtained by chemical or enzymatic hydrolysis of at least one fatty acid triglyceride typically resulting from a vegetable oil. Alternatively, it can result from an animal fat. For its part, the ester of the acid can be a triglyceride of the acid or it can be obtained by esterification of the acid or transesterification of a triglyceride using a monoalcohol. Examples of mono- or polyunsaturated aliphatic carboxylic acid esters comprise linear or branched C₁-C₆ alkyl esters, such as the methyl, ethyl, propyl, isopropyl, butyl, tert-butyl, pentyl, isopentyl or hexyl esters, without this list being limiting. An example of triglyceride is triolein.

Preferably, oleic acid, palmitoleic acid, erucic acid, linoleic acid, α-linolenic acid, their mixtures and/or one of their esters, more preferentially oleic acid or one of its esters, in particular methyl oleate, is/are used.

Mention may in particular be made, as examples of vegetable oils, of wheatgerm, sunflower, argan, hibiscus, coriander, grape seed, sesame, corn, apricot, castor, shea, avocado, olive, peanut, soybean, sweet almond, palm, rapeseed, cottonseed, hazelnut, macadamia, jojoba, alfalfa, poppy, red kuri squash, sesame, pumpkin, blackcurrant, evening primrose, lavender, borage, millet, barley, quinoa, rye, safflower, candlenut, passionflower, musk rose, echium, camelina or camellia oil. Alternatively, one or more oils resulting from biomass of microalgae can be used.

Examples of mono- and dicarboxylic acids (and their esters) capable of being obtained by oxidative cleavage of the abovementioned acids are collated in the table below.

| Unsaturated acids | Monoacids | Diacids |
|---|---|---|
| Oleic | Pelargonic | Azelaic |
| Linoleic | Hexanoic | Azelaic, malonic |
| Arachidonic | Hexanoic | Malonic, pentanedioic |
| Palmitoleic | Heptanoic | Azelaic |
| Linolenic | Propionic | Azelaic, malonic |
| Ricinoleic | 3-Hydroxynonanoic | Azelaic |
| Erucic | Pelargonic | Brassylic |

It is understood that, in the light of the abundance of the unsaturated fatty acids above, the dicarboxylic acid obtained according to one embodiment of the invention is preferably azelaic acid.

In another embodiment of the invention, at least one non-functionalized cyclic olefin is used as substrate.

Examples of non-functionalized cyclic olefins which can be used as substrate in this invention comprise: cyclohexene, cycloheptene, cyclooctene, cyclononene, cyclodecene, cycloundecene, cyclododecene, dicyclopentadiene, norbornene and norbornadiene, without this list being limiting.

Examples of dicarboxylic acids (and their esters) capable of being obtained by oxidative cleavage of non-functionalized cyclic olefins are collated in the table below.

| Cyclic olefin | Diacids |
|---|---|
| Cyclohexene | Adipic acid |
| Cycloheptene | Pimelic acid |
| Cyclooctene | Suberic acid |
| Cyclononene | Azelaic acid |
| Cyclodecene | Sebacic acid |
| Cycloundecene | Undecanedioic acid |
| Cyclododecene | Dodecanedioic acid |

Tricarboxylic acids can be obtained by oxidative cleavage of cyclic olefins exhibiting a constrained cyclic structure, in particular a bridged structure, such as norbornene, dicyclopentadiene or norbornadiene. For example, the oxidative cleavage of norbornene makes it possible to obtain butane-1,2,4-tricarboxylic acid.

In the continuation of this description, the term “substrate” will be used for the sake of simplicity to refer both to functionalized or non-functionalized linear olefins and to non-functionalized cyclic olefins which can be reacted in the process according to the invention. In this process, the chosen substrate is oxidized using hydrogen peroxide in the presence of a specific catalyst.

The amount of hydrogen peroxide used in this process is generally between 4 and 20 molar equivalents, preferably between 4 and 10 molar equivalents and better still between 4 and 8 molar equivalents, limits included, of hydrogen peroxide per one molar equivalent of double bond present within the substrate. In the case where the substrate consists of a mixture of olefins, in particular of a mixture of functionalized linear olefins, as in the case of vegetable oils, the number of double bonds can be calculated by referring to the acid numbers and iodine numbers of these fatty substances, for example according to the Wijs method with iodine monochloride.

Hydrogen peroxide can be used at a concentration of between 1% and 70% (w/V), preferably between 30% (w/V) and 70% (w/V), limits included, preferably at 60% (w/V).

The catalyst used in the process according to the invention consists of at least one onium halooxodiperoxometallate. The onium can be chosen from a tetraalkylammonium, a tetraalkylphosphonium and an alkylpyridinium, the alkyl groups of which independently include from 1 to 20 carbon atoms (preferably from 1 to 18 carbon atoms), benzethonium and triphenylphosphoranylidene. In the present invention, it is preferred to use a tetraalkylammonium.

Examples of onium ions which can be used according to the invention are in particular: dodecyltrimethylammonium, trioctylmethylammonium, tetradecyltrimethylammonium, hexadecyltrimethylammonium, dimethyldihexadecylammonium, octadecyltrimethylammonium, dioctadecyldimethylammonium, benzyldimethyldodecylammonium, benzyldimethyltetradecylammonium, benzyldimethylhexadecylammonium, benzyldimethyloctadecylammonium, dodecylpyridinium, hexadecylpyridinium, benzethonium, tetrabutylammonium, tetradecyltrihexylphosphonium, hexadecyltributylphosphonium, bis(triphenylphosphoranylidene)ammonium and tetrabutylammonium.

For its part, the halooxodiperoxometallate can be chosen from the compounds of formula (I):

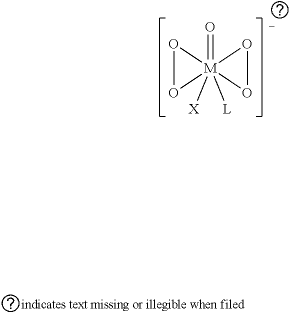

where:

M is a metal chosen from W and Mo,

X is a halogen atom,

L denotes a neutral ligand having at least one non-bonding lone pair.

Examples of ligands L are water, amines, ethers and phosphines, without this list being limiting. It is preferred, according to this invention, for L to be H₂O.

It is furthermore preferred to use halooxodiperoxotungstates (M=W) such as chlorooxodiperoxotungstate, fluorooxodiperoxotungstate, bromooxodiperoxotungstate and iodooxodiperoxotungstate, more preferentially chlorooxodiperoxotungstate.

The catalyst used according to the invention can be prepared as described by Ryo Ishimoto et al. in Chem. Lett., 2013, 42, 476-478. Alternatively, it can be synthesized according to a process comprising:

(a) a first stage in which an aqueous solution of a metal salt, preferably an optionally hydrated tungstic or molybdic acid salt, in particular an optionally hydrated alkali metal salt, is brought into contact with a strong acid and hydrogen peroxide, in the presence of a molecule L defined above, and

(b) a second stage consisting in reacting the product resulting from the first stage with an aqueous solution of an onium halide.

The catalyst can subsequently be isolated:

(c1) either by cooling the mixture in order to precipitate the catalyst, which can subsequently be recovered by filtration and then optionally rinsed with water and/or using an alcohol, such as ethanol,

(c2) or by separation of the organic phase and of the aqueous phase which are obtained, extraction of the aqueous phase using a water-immiscible solvent, such as dichloromethane, ethyl acetate, cyclohexane, toluene or methyl tert-butyl ether, in order to obtain a second organic phase, then drying using a dehydrating agent, such as anhydrous sodium sulfate, and finally evaporation under vacuum of the combined organic phases.

In the first stage above, it is preferred to use sodium tungstate dihydrate as metal salt and sulfuric acid as strong acid.

The amount of strong acid is adjusted so as to bring the pH of the reaction medium to a value of between 0.5 and 2.0, preferably between 1.0 and 1.5. The hydrogen peroxide is preferably used in an amount representing from 1 to 10 molar equivalents, more preferentially from 2 to 10 molar equivalents, better still from 3 to 8 molar equivalents, indeed even from 5 to 6 molar equivalents, with respect to the molar amount of metal acid salt.

Examples of onium halides which can be used in the second stage of this process are in particular: dodecyltrimethylammonium chloride, trioctylmethylammonium chloride, tetradecyltrimethylammonium chloride, hexadecyltrimethylammonium chloride, dimethyldihexadecylammonium chloride, octadecyltrimethylammonium chloride, dioctadecyldimethylammonium chloride, benzyldimethyldodecylammonium chloride, benzyldimethyltetradecylammonium chloride, benzyldimethylhexadecylammonium chloride, benzyldimethyloctadecylammonium chloride, dodecylpyridinium chloride, hexadecylpyridinium chloride, benzethonium chloride, tetrabutylammonium chloride, tetradecyltrihexylphosphonium chloride, hexadecyltributylphosphonium chloride, bis(triphenylphosphoranylidene)ammonium chloride, terabutylammonium fluoride and their mixtures. Generally, the above chloride salts can be replaced by fluoride, bromide or iodide salts of the same cations.

In addition, the onium halide added in the second stage is advantageously used in an equimolar amount with respect to the metal acid salt.

The amount of catalyst used in the process according to the invention is generally of between 0.1 molar % and 10 molar %, preferably between 0.5 molar % and 8 molar %, more preferentially between 2 molar % and 6 molar %. with respect to the molar amount of double bonds present within the substrate. Alternatively or in addition, it can represent from 0.1% to 15% by weight, preferably from 5% to 10% by weight, with respect to the molar amount of double bonds present within the substrate. The process according to the invention can thus be implemented under economically very advantageous conditions, insofar as it uses a small amount of catalyst, which can moreover be easily manufactured. In addition, the absence of toxic metals in this catalyst makes it possible to carry out this process under conditions which are friendlier to the environment and to human health than some of the processes of the prior art.

As some of the catalysts prepared as described above are novel, another subject-matter of the invention is these catalysts, of formula (II):

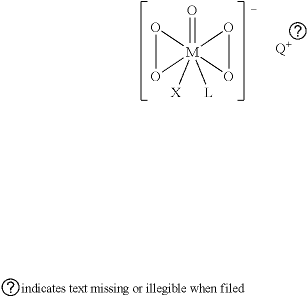

in which:

M is a metal chosen from W and Mo,

X is a halogen atom,

L denotes a neutral ligand having at least one non-bonding lone pair,

Q⁺ denotes an onium cation of formula N⁺(R₁R₂R₃R₄), where:
R₁ denotes a linear or branched, preferably linear, C₆-C₂₀ (for example C₁₂-C₁₈) alkyl group, R₂ and R₃ each independently denote a linear or branched, preferably linear, C₁-C₄ alkyl group and R₄ denotes a linear or branched, preferably linear, C₁-C₄ alkyl group or an aryl group, or else
R₁ denotes a linear or branched, preferably linear, C₄-C₁₄ alkyl group and R₂, R₃ and R₄ form, with the nitrogen atom, a pyridinium group.

Examples of ligands L are water, amines, ethers and phosphines; preferably, L is H₂O.

In a specific embodiment, R₁ denotes a linear or branched, preferably linear, C₁₂-C₁₈ (for example C₁₂-C₁₄) alkyl group, R₂ and R₃ each independently denote a linear or branched, preferably linear, C₁-C₄ alkyl group and R₄ denotes a linear or branched, preferably linear, C₁-C₄ alkyl group or an aryl group.

In another specific embodiment, R₁ denotes a linear or branched, preferably linear, C₆-C₂₀ (for example C₁₂-C₁₄) alkyl group, R₂ and R₃ each denote a methyl group and R₄ denotes a methyl group or an aryl group.

In a preferred embodiment of the invention, R₁ denotes a linear or branched, preferably linear, C₁₂-C₁₈ (for example C₁₂-C₁₄) alkyl group, R₂ and R₃ each denote a methyl group and R₄ denotes a methyl group or an aryl group.

In the case where the linear olefin is a mono- or polyunsaturated aliphatic carboxylic acid, or one of its esters, the novel catalysts above make it possible to obtain the desired dicarboxylic acid with a molar yield of at least 40%, preferably of at least 50%, at least 60%, at least 70%, indeed even at least 80% or even at least 90%.

Furthermore, the halogen is preferably chlorine or fluorine, more preferentially chlorine. Examples of catalysts corresponding to the above definition are given above. Among these, dodecyltrimethylammonium chlorooxodiperoxotungstate is preferred for its ease of preparation without organic solvent and its efficiency.

The oxidative cleavage process according to the invention generally comprises the stages consisting in:
mixing the catalyst, previously formed in situ or isolated, with the substrate, optionally brought beforehand to a temperature of 20 to 120° C., and with hydrogen peroxide, preferably at ambient temperature,
bringing the mixture to a temperature of from 20 to 120° C., preferably from 50 to 120° C., more preferentially from 50 to 100° C., or better still from 80 to 100° C., with stirring, for example at 50-1200 revolutions/min, for a period of time which can, for example, range from 2 to 24 hours, in particular from 4 to 6 hours, and
recovering the products thus formed by any means, in particular by crystallization, filtration, distillation, liquid-liquid extraction or chromatographic purification.

In a specific embodiment, the substrate consists of at least one monounsaturated linear olefin, and is employed in the preparation of at least one monocarboxylic acid. In such a specific embodiment, the olefin is non-functionalized, or else functionalized by any group other than a carboxyl.

In another specific embodiment of the invention, the substrate consists of at least one cyclic olefin (such as cyclohexene, cycloheptene, cyclooctene, cyclononene, cyclodecene, cycloundecene, cyclododecene, dicyclopentadiene, norbornene and norbornadiene, without this list being limiting), and is employed in the preparation of at least one di- or tricarboxylic acid or one of its esters.

In a preferred embodiment of the invention, the substrate consists of at least one mono- or polyunsaturated aliphatic carboxylic acid or one of its esters, and is employed in the preparation of at least one dicarboxylic acid or one of its esters, and optionally of at least one monocarboxylic acid.

In this preferred embodiment, the oxidative cleavage process according to the invention generally comprises the stages consisting in:
mixing the catalyst, previously formed in situ or isolated, with the substrate, optionally brought beforehand to a temperature of 20 to 120° C., and with hydrogen peroxide, preferably at ambient temperature,
bringing the mixture to a temperature of from 20 to 120° C., preferably from 50 to 120° C., more preferentially from 50 to 100° C., or better still from 80 to 100° C., with stirring, for example at 50-1200 revolutions/min, for a period of time which can, for example, range from 2 to 24 hours, in particular from 4 to 6 hours, and
recovering the dicarboxylic acid or its ester thus formed, and optionally the monocarboxylic acid or its ester obtained as coproduct.

Advantageously, this process does not use an organic solvent, in particular chosen from 1,2-dichloroethane, dichloromethane, chloroform, ethyl ether, tert-butanol or acetonitrile.

The dicarboxylic acid can be recovered by crystallization, followed by filtration or centrifugation. To do this, the reaction mixture can be cooled, for example to 0-30° C. and preferably to 15-25° C., in order to precipitate the dicarboxylic acid. The latter can subsequently be optionally redissolved in water and then precipitated from an appropriate solvent, in particular a non-polar organic solvent, such as heptane, which makes it possible to extract the monocarboxylic acid formed simultaneously.

Examples of dicarboxylic acids which can be prepared according to this preferred embodiment of the invention are in particular azelaic acid, adipic acid, succinic acid, sebacic acid, 1,7-heptanedioic acid, 1,8-octanedioic acid, 1,11-undecanedioic acid, 1,12-dodecanedioic acid, brassylic acid, 1,14-tetradecanedioic acid, 1,15-pentadecanedioic acid and thapsic acid, preferably azelaic acid, adipic acid, succinic acid, sebacic acid, 1,12-dodecanedioic acid, brassylic acid and thapsic acid, better still azelaic acid.

These dicarboxylic acids can be used in particular as monomer in the manufacture of polymers, such as polyesters or polyamides, as plasticizer, in the manufacture of esters or of lubricants or as cosmetic or dermatological active agent. When the dicarboxylic acid consists of azelaic acid, it can additionally be used as antibacterial active agent intended in particular for the treatment of acne or rosacea.

The mono-, di- or tricarboxylic acids prepared by the process of the invention can subsequently be reduced to give alcohols, for example by means of lithium aluminum hydride, as described in Biomacromolecules, 2010, 11, 911-918 (reduction of azelaic acid to give 1,9-nonanediol), or by metallo-catalyzed hydrogenation (Chem. Commun., 2018, 54, 13319).

### FIGURES

FIG. 1 represents the molecular structure of the compound described in example 2, obtained by XRD and represented with 50% probability ellipsoids.

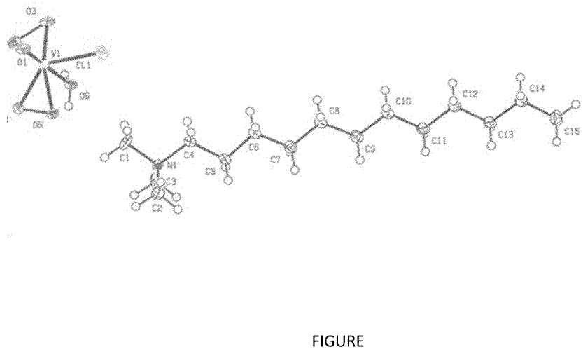

### EXAMPLES

A better understanding of the invention will be obtained in the light of the following examples, which are given purely by way of illustration and do not have the aim of limiting the scope of the invention, defined by the appended claims.

Materials and Methods

The reactants originate from ordinary commercial suppliers (Sigma-Aldrich-Merck, Acros, Alfa-Aesar, Fisher) and were used without prior purification.

All the reactions were carried out in air, at atmospheric pressure.

The GC-MS analyses were carried out with a Shimadzu QP2010SE instrument, using H₂ as carrier gas, with a Zebron Fast GC (Phenomenex) (20 m×0.18 mm×0.18 m) column. The GC-MS quantification was carried out using octanoic acid as internal standard. The concentrations of azelaic acid, pelargonic acid and oleic acid were calculated using a calibration curve (R²>0.99 in the three cases).

The proton Nuclear Magnetic Resonance (NMR) spectra were recorded on an Avance 400 NMR spectrometer at 400.1 MHz (Bruker) at 25° C. The chemical shifts are expressed in ppm (parts per million) with respect to the signal of the residual non-deuterated solvent. The multiplicity of the signals is described as follows: singlet (s), doublet (d), triplet (t) and multiplet (m).

### Example 1: General Process for the Preparation and Characterization of the Catalysts

1A) Preparation by Precipitation (Case of the Water-Insoluble Ammoniums, Such as Dodecyltrimethylammonium Chloride and Hexadecylpyridinium Chloride):

Na₂WO₄.2H₂O (6.93 mmol, 1.00 eq.) is introduced into a 50 ml round-bottomed flask and then 5 ml of distilled water are added in order to dissolve Na₂WO₄.2H₂O. An H₂SO₄ solution (2M, 5 mmol; 0.72 eq.) is subsequently added to this solution, immediately followed by the addition of aqueous hydrogen peroxide solution (30 w/v %; 37.48 mmol; 5.4 eq.). The solution turns yellow and then virtually colorless. The pH of the latter is located between 0.9 and 1.1; if not, it can be adjusted with a few additional drops of the H₂SO₄ solution. The alkylammonium chloride, dissolved beforehand in 5 ml of distilled water, is subsequently added dropwise (7.28 mmol; 1.05 eq.). The medium is subsequently stirred at 20° C. for 30 minutes, then placed under cold conditions (4° C.) overnight. The precipitate formed is filtered off and then rinsed with H₂O (4×50 ml) and then with ethanol cooled to 0° C. (25 ml). The product is subsequently predried on a rotary evaporator and then dried overnight under vacuum in the presence of P₂O₅.

1B) Preparation by Extraction (Case of all the Other Ammoniums and Phosphoniums, Also Applicable to Those Prepared by Precipitation):

Na₂WO₄.2H₂O (6.93 mmol; 1.0 eq.) is introduced into a 50 ml round-bottomed flask and then 5 ml of distilled water are added in order to dissolve Na₂WO₄.2H₂O. An H₂SO₄ solution (2M, 5 mmol; 0.72 eq.) is subsequently added to this solution, immediately followed by the addition of aqueous hydrogen peroxide solution (30 w/v %; 37.48 mmol; 5.4 eq.). The solution turns yellow and then virtually colorless. The pH of the latter is located between 0.9 and 1.1; if not, it can be adjusted with a few additional drops of the H₂SO₄ solution. The alkylammonium halide, in solution in 10 ml of dichloromethane, is subsequently added to the medium (7.28 mmol; 1.05 eq.). The solution is subsequently stirred vigorously at 20° C. for 1 h 30. The phases are subsequently separated and the aqueous phase is extracted with 15 ml of dichloromethane. The organic phase is dried with anhydrous sodium sulfate and then evaporated on a rotary evaporator. The solid obtained is dried under vacuum overnight.

The yields obtained on conclusion of the above processes are collated in the following table.

| | Isolated |
|---|---|
| Halooxodiperoxometallate catalyst | yield |
| Dodecyltrimethylammonium chlorooxodiperoxotungstate | 85% |
| Trioctylmethylammonium chlorooxodiperoxotungstate | 66% |
| Tetradecyltrimethylammonium chlorooxodiperoxotungstate | 34% |
| Octadecyltrimethylammonium chlorooxodiperoxotungstate | 86% |
| Dimethyldioctadecylammonium chlorooxodiperoxotungstate | 47% |
| Tetrabutylammonium chlorooxodiperoxotungstate | 62% |
| Benzyldimethyldodecylammonium chlorooxodiperoxotungstate | 36% |
| Benzyldimethyltetradecylammonium chlorooxodiperoxotungstate | 60% |
| Benzyldimethylhexadecylammonium | 52% |
| chlorooxodiperoxotungstate | |
| Benzyldimethylstearylammonium chlorooxodiperoxotungstate | 79% |
| Dodecylpyridinium chlorooxodiperoxotungstate | 58% |
| Hexadecylpyridinium chlorooxodiperoxotungstate | 75% |
| Benzethonium chlorooxodiperoxotungstate | 58% |
| Tetradecyltrihexylphosphonium chlorooxodiperoxotungstate | 62% |
| Bis(triphenylphosphoranylidene)ammonium | 68% |
| chlorooxodiperoxotungstate | |
| Tetrabutylammonium fluorooxodiperoxotungstate | |

### Example 2: Preparation and Characterization of Dodecyltrimethylammonium Chlorooxodiperoxotungstate

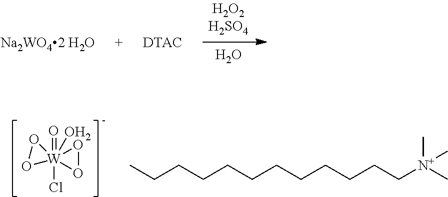

Na₂WO₄.2H₂O (6.93 mmol, 1.00 eq.) is introduced into a 50 ml round-bottomed flask and then 5 ml of distilled water are added in order to dissolve Na₂WO₄.2H₂O. An H₂SO₄ solution (2M, 5 mmol; 0.72 eq.) is subsequently added to this solution, immediately followed by the addition of aqueous hydrogen peroxide solution (30 w/v %; 37.48 mmol; 5.4 eq.). The solution turns yellow and then virtually colorless. The pH of the latter is located between 0.9 and 1.1; if not, it can be adjusted with a few additional drops of the H₂SO₄ solution.

Dodecyltrimethylammonium chloride, dissolved beforehand in 5 ml of distilled water, is subsequently added dropwise (7.28 mmol; 1.05 eq.). A white precipitate forms, then redissolves. The medium is subsequently stirred at 20° C. for 30 minutes, then placed under cold conditions (4° C.) overnight. The precipitate formed is filtered off and then rinsed with H₂O (4×50 ml) and then with ethanol cooled to 0° C. (25 ml). The product is subsequently predried on a rotary evaporator and then dried overnight under vacuum in the presence of P₂O₅.

Dodecyltrimethylammonium chlorooxodiperoxotungstate is obtained in the form of a white powder with a molar yield of 85%.

¹H NMR (CDCl₃, 400 MHz) δ: 0.75 (m, 3H); 1.06-1.30 (m, 18H); 1.60 (m, 2H); 3.06-3.20 (s, 9H); 3.81 (m, 2H); ¹³C NMR (101 MHz, CDCl₃) δ: 66.8, 52.9, 31.8, 29.5, 29.4, 29.2, 26.2, 23.0, 22.5, 13.9; IR νₘₐₓ 2915, 2850, 1469, 947, 835, 775, 720, 619, 572, 547, 486, 419.

Single-Crystal X-Ray Diffraction:

The measurements were carried out on a D8 VENTURE Bruker AXS diffractometer equipped with a PHOTON 100 (CMOS) detector, with Mo-Kα radiation (λ=0.71073 Å, multilayer monochromator), T=150(2) K; monoclinic crystal P 2₁/c (IT. #14), a=18.8525(16), b=7.3078(7), c=15.3995(14) Å, β=97.340(4)°, V=2104.2(3) Å³. Z=4, d=1.723 g·cm⁻³, μ=5.643 mm⁻¹. The structure was solved by a dual space algorithm using the SHELXT program [G. M. Sheldrick, Acta Cryst., A71 (2015), 3-8], then refined by full matrix least squares methods based on F2 (SHELXL) [Sheldrick G. M., Acta Cryst., C71 (2015), 3-8]. All the atoms other than hydrogen were refined with anisotropic atomic shift parameters. A final refinement on F2 with 4810 unique intensities and 227 parameters converged to ωR(F2)=0.0496 (R(F)=0.0218) for 4125 reflections observed with I>2σ(I).

The structure obtained is illustrated in the appended FIGURE.

### Example 3: Oxidative Cleavage of Oleic Acid

A 250 ml single-necked round-bottomed flask equipped with a 20×10 mm magnetic bar is charged with dodecyltrimethylammonium chloroperoxotungstate catalyst (700 mg, 1.28 mmol, 0.040 eq.) and then with oleic acid (90% purity) (10.0 g, 31.86 mmol, 1.0 eq.). The mixture is stirred at 300 revolutions/min at 22° C. for 5 min, forming a homogeneous white liquid phase. 60% (w/V) aqueous hydrogen peroxide solution (10.84 ml, 191.16 mmol, 6.0 eq.) is then added dropwise to this mixture at 22° C. with stirring over 5 min. The round-bottomed flask is subsequently equipped with a reflux condenser and the reaction mixture is brought to reflux by contact with a metal heating block (DrySyn block, Asynt) preheated to 90° C., with stirring at 1000 revolutions/min, for 5 h. During the reaction, the reaction medium remains two-phase, with a white lower phase and a colorless upper phase. On completion of the reaction, the medium is allowed to cool to 22° C. After cooling, a white solid appears at the bottom of the round-bottomed flask.

A GC-MS analysis of the medium is carried out after derivatization with trimethylsulfonium hydroxide (0.2 mol/l in methanol), according to a procedure described in Journal of Chromatography A, 2004, 1047, 111-116. The molar yields are then calculated with the help of a calibration curve, using octanoic acid as internal standard: azelaic acid 98%, pelargonic acid 74%.

The azelaic acid can be isolated by virtue of the following procedure: on completion of the reaction, 25 ml of deionized water are added to the round-bottomed reaction flask and then the mixture is heated to 90° C., with stirring at 300 revolutions/min. After heating for 10 min, the white solid dissolves completely, thus giving an off white solution. 20 ml of heptane are then added to the mixture and stirring is continued at 90° C. for 10 min. The heating and the stirring are subsequently stopped and the medium is then allowed to cool to 22° C. After 3 h, a white solid appears at the bottom of the round-bottomed flask, the upper phase being colorless. This mixture is then filtered on a Whatman glass microfiber disc (4.25 cm in diameter, reference 1820042) and rinsed with 3×75 ml of heptane. The white solid, consisting of azelaic acid, is collected and dried under reduced pressure in a desiccator, in the presence of P₂O₅. A weight of 6.44 g is obtained. The filtrate can be evaporated under reduced pressure, to give pelargonic acid in the form of a colorless oil.

The purity of the azelaic acid obtained is calculated by GC-MS analysis after derivatization with N,O-bis(trimethylsilyl)trifluoroacetamide with 1% of trimethylchlorosilane: 50 mg of azelaic acid are dissolved in 1 ml of THF, then 10 μl of this solution are introduced into a GC vial, followed by the addition of 100 μl of anhydrous pyridine and then 100 μl of N,O-bis(trimethylsilyl)trifluoroacetamide with 1% of trimethylchlorosilane. The mixture is heated and stirred in the GC vial at 40° C. for 1 h, then diluted with 600 μl of THF and injected in GC-MS. After GC-MS analysis, a purity of 91% is determined for the azelaic acid, the remainder consisting of traces of pelargonic acid (7%) and of C₄ impurities (2%).

Taking into account the calculated purities and the weight of azelaic acid collected, the corrected isolated molar yield of azelaic acid is 97%.

By following the same protocol as in example 2 but with the other catalysts, the following yields are obtained:

| | Azelaic | Pelargonic |
|---|---|---|
| | acid yield | acid yield |
| Catalyst | (%) | (%) |
| [WO(O₂)₂Cl•H₂O][tetradecyltrimethylammonium] | 78% | 72% |
| [WO(O₂)₂Cl•H₂O][octadecyltrimethylammonium] | 69% | 67% |
| [WO(O₂)₂Cl•H₂O][trioctylmethylammonium] | 39% | 38% |
| [WO(O₂)₂Cl•H₂O][dimethyldioctadecylammonium] | 39% | 38% |
| [WO(O₂)₂Cl•H₂O][benzyldimethyldodecylammonium] | 71% | 68% |
| [WO(O₂)₂Cl•H₂O][benzyldimethyltetradecylammonium] | 68% | 68% |
| [WO(O₂)₂Cl•H₂O][benzyldimethylhexadecylammonium] | 71% | 66% |
| [WO(O₂)₂Cl•H₂O][benzyldimethylstearylammonium] | 68% | 69% |
| [WO(O₂)₂Cl•H₂O][dodecylpyridinium] | 62% | 59% |
| [WO(O₂)₂Cl•H₂O][hexadecylpyridinium] | 70% | 66% |
| [WO(O₂)₂Cl•H₂O][benzethonium] | 64% | 62% |
| [WO(O₂)₂Cl•H₂O][tetradecyltrihexylphosphonium] | 39% | 40% |
| [WO(O₂)₂Cl•H₂O][bis(triphenylphosphoranylidene)ammonium] | 55% | 54% |
| [WO(O₂)₂Cl•H₂O][tetrabutylammonium] | 64% | 59% |
| [WO(O₂)₂F•H₂O][tetrabutylammonium] | 40% | 38% |
| [MoO(O₂)₂Cl•H₂O][dodecyltrimethylammonium] | 40% | 37% |

### Example 4: Oxidative Cleavage of Cyclohexene

A reactor (external diameter 16 mm, 15 ml) equipped with a 10×5 mm magnetic bar is charged with dodecyltrimethyl ammonium chlorooxodiperoxotungstate catalyst (44.7 mg; 0.08 mmol; 0.029 eq.) and then with cyclohexene (9900 purity) (234.9 mg; 2.83 mmol; 1.0 eq.). 600% (w/V) aqueous hydrogen peroxide solution (960 al; 16.93 mmol; 5.9 eq.) is then added to this mixture.

The reaction mixture is heated by contact with a metal heating block (DrySyn block, Asynt) already preheated to 90° C., with stirring at 1000 revolutions/min, for 5 h. During the reaction, the reaction medium becomes monophasic and completely clear. On completion of the reaction, the medium is allowed to cool to 25° C., letting a white solid appear at the bottom of the reactor.

Analysis of the reaction and quantification by ¹H NMR: an internal standard, 1,4-dibromobenzene (669.5 mg; 2.83 mmol), is then added to a 25 ml volumetric flask and then the entire reaction medium is homogenized with d6-DMSO before being added to the volumetric flask. The latter is then made up to volume with deuterated dichloromethane until the internal standard has completely dissolved and then with d6-DMSO up to the graduation mark. ¹H NMR analysis of this mixture is carried out and makes it possible to calculate a 90% molar yield of adipic acid.

1-17. (canceled)

18. A process for the oxidative cleavage of a substrate consisting of at least one functionalized or non-functionalized linear olefin or of at least one non-functionalized cyclic olefin, comprising converting a carbon-carbon double bond of the substrate into two separate oxidized functional groups chosen from aldehydes, ketones and carboxylic acids, using hydrogen peroxide, in the presence of a metal catalyst, characterized in that the catalyst is formed of at least one onium halooxodiperoxometallate.

19. The process of claim 18, characterized in that the onium is selected from the group consisting of a tetraalkylammonium, a tetraalkylphosphonium and an alkylpyridinium, the alkyl groups of which independently include from 1 to 20 carbon atoms, benzethonium and triphenylphosphoranylidene.

20. The process of claim 18, characterized in that the halooxodiperoxometallate is a compound of formula (I): 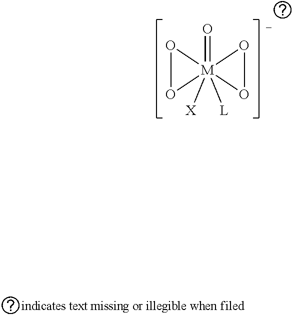
where:
M is a metal selected from the group consisting of W and Mo,
X is a halogen atom,
L denotes a neutral ligand having at least one non-bonding lone pair.

21. The process of claim 20, characterized in that L is selected from the group consisting of water, amines, ethers and phosphines.

22. The process of claim 21, characterized in that L is water.

23. The process of claim 20, characterized in that the halooxodiperoxometallate is selected from the group consisting of chlorooxodiperoxotungstate, fluorooxodiperoxotungstate, bromooxodiperoxotungstate and iodooxodiperoxotungstate.

24. The process of claim 23, characterized in that the halooxodiperoxometallate is chlorooxodiperoxometallate.

25. The process of claim 18, characterized in that the catalyst is employed in an amount ranging from 0.1 molar % to 10 molar %, with respect to the molar amount of double bonds in the substrate.

26. The process of claim 25, characterized in that the catalyst is employed in an amount ranging from 2 molar % to 6 molar %, with respect to the molar amount of double bonds in the substrate.

27. The process of claim 18, characterized in that the hydrogen peroxide is employed in an amount ranging from 4 to 20 molar equivalents, with respect to the molar amount of double bonds in the substrate.

28. The process of claim 18, characterized in that the substrate consists of at least one mono- or polyunsaturated aliphatic carboxylic acid or one of its esters, and in that it is employed in the preparation of at least one dicarboxylic acid or one of its esters, respectively, and optionally of at least one monocarboxylic acid.

29. The process of claim 28, characterized in that the mono- or polyunsaturated aliphatic carboxylic acid or one of its esters is selected from the group consisting of: oleic acid, palmitoleic acid, erucic acid, linoleic acid, α-linolenic acid, their mixtures and/or one of their esters.

30. The process of claim 29, characterized in that the mono- or polyunsaturated aliphatic carboxylic acid or one of its esters is oleic acid or one of its esters.

31. The process of claim 28, characterized in that the dicarboxylic acid is selected from the group consisting of azelaic acid, adipic acid, succinic acid, sebacic acid, 1,7-heptanedioic acid, 1,8-octanedioic acid, 1,11-undecanedioic acid, 1,12-dodecanedioic acid, brassylic acid, 1,14-tetradecanedioic acid, 1,15-pentadecanedioic acid and thapsic acid.

32. The process of claim 31, characterized in that the dicarboxylic acid is azelaic acid.

33. The process of claim 28, characterized in that the method comprises:
mixing the catalyst, previously formed in situ or isolated, with the substrate, optionally brought beforehand to a temperature of 20 to 120° C., and with hydrogen peroxide,
bringing the mixture to a temperature of from 20 to 120° C. for a period of time ranging from 2 to 24 hours, with stirring, and
recovering the dicarboxylic acid or its ester thus formed, and optionally the monocarboxylic acid or its ester obtained as coproduct.

34. The process of claim 18, characterized in that the substrate consists of at least one non-functionalized cyclic olefin.

35. The process of claim 34, characterized in that the substrate is selected from the group consisting of: cyclohexene, cycloheptene, cyclooctene, cyclononene, cyclodecene, cycloundecene, cyclododecene, dicyclopentadiene, norbornene and norbornadiene.

36. A catalyst of formula (II): 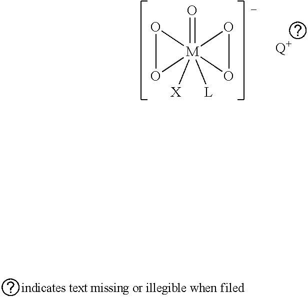
in which:
M is a metal chosen from W and Mo,
X is a halogen atom,
L denotes a neutral ligand having at least one non-bonding lone pair.
Q⁺ denotes an onium cation of formula N⁺(R₁R₂R₃R₄), where:
R₁ denotes a linear or branched C₆-C₂₀ alkyl group, R₂ and R₃ each independently denote a linear or branched C₁-C₄ alkyl group and R₄ denotes a linear or branched, C₁-C₄ alkyl group or an aryl group, or else
R₁ denotes a linear or branched C₄-C₁₄ alkyl group and R₂, R₃ and R₄ form, with the nitrogen atom, a pyridinium group.

37. The catalyst of claim 36, characterized in that R₁ denotes a linear or branched C₁₂-C₁₈ alkyl group, R₂ and R₃ each denote a methyl group and R₄ denotes a methyl group or an aryl group.

38. The catalyst of claim 36, characterized in that the halogen is chlorine or fluorine.

39. The catalyst of claim 36, characterized in that the catalyst is dodecyltrimethylammonium chlorooxodiperoxotungstate.

40. The catalyst of claim 36, characterized in that L is H₂O.

41. The catalyst of claim 36, characterized in that when R₁, R₂ and/or R₃ denotes an alkyl group, this alkyl group is linear.

42. A method for the oxidative cleavage of compounds selected from the group consisting of mono- or polyunsaturated aliphatic carboxylic acids and their esters, comprising contacting said compounds with hydrogen peroxide in the presence of the catalyst of claim 36.
